(19) United States
(12) Patent Application Publication
(10) Pub. No.: US 2010/0177281 A1
(43) Pub. Date: Jul. 15, 2010

(54) EYE DISORDER DETECTION CHART

(76) Inventor: Abbas M. Husain, Merchantville, NJ (US)

Correspondence Address: PATWRITE LLC, 408 W. MAIN ST., MARSHALLTOWN, IA, 50158-5759, US

(21) Appl. No.: 12/683,898

(22) Filed: Jan. 7, 2010

Related U.S. Application Data
(60) Provisional application No. 61/142,956, filed on Jan. 7, 2009.

(51) Int. Cl. A61B 3/02

(52) U.S. Cl. 351/239; 351/246

(57) ABSTRACT

An eye chart comprises a series of sinusoidal lines spaced at intervals of a selected to discriminate between normal vision and vision impaired by the presence of a cataract. Groups of sinusoidal lines have progressively different distances between the series with one series representing normal vision with the space between the lines growing progressively further apart to indicate the advancement of the cataract. A user counts the number of lines of the group that is clearly distinct. A user with normal vision will be able to count the number of lines, while a person with cataracts will require the space between the lines to be further apart in order to count depending on the stage of cataract development. A light source may be used to indicate night vision problems. A practitioner can record the progression of cataracts by keeping track of any changes over time.

This application claims priority and herein incorporates by reference U.S. provisional patent application 61/142,956, filed Jan. 7, 2009.

### BACKGROUND OF THE INVENTION

Cataracts account for 48% of world blindness or over 18 million people have some cataract development according to the World Health Organization (WHO). In the United States, 42 percent of people between the ages of 52 to 64 have some cataract development, 60% for those between 65 to 74 and 91% for those between the ages of 75 to 85.

There are many causes of cataracts; long-term ultraviolet exposure, radiation, diabetes, hypertension with the most common cause being age. The most successful treatment for cataracts is surgery to remove the occluded lens. Currently, only a thorough eye examination can detect the early onset of a cataract. There is a need for an easy to use screening detection device that enables an early diagnosis of cataracts.

### SUMMARY OF THE INVENTION

An eye chart comprises a series of sinusoidal lines spaced at intervals of a selected to discriminate between normal vision and vision impaired by the presence of a cataract. Groups of sinusoidal lines have progressively different distances between the series with one series representing normal vision with the space between the lines growing progressively further apart to indicate the advancement of the cataract. A user counts the number of lines of the group that is clearly distinct. A user with normal vision will be able to count the number of lines, while a person with cataracts will require the space between the lines to be further apart in order to count depending on the stage of cataract development. A light source may be used to indicate night vision problems. A user can record the progression of the cataracts by keeping track over time any changes.

Other features and advantages of the instant invention will become apparent from the following description of the invention which refers to the accompanying drawings.

### BRIEF DESCRIPTION OF THE DRAWINGS

FIG. 1 is an eye chart for cataract detection according to an embodiment of the present invention.

FIG. 2 is an eye chart for cataract detection according to an embodiment of the present invention.

FIG. 3 is an eye chart for cataract detection according to an embodiment of the present invention.

FIG. 4 is an eye chart for cataract detection according to an embodiment of the present invention.

FIG. 5 is an eye chart for cataract detection according to an embodiment of the present invention.

FIG. 6 is an eye chart for cataract detection according to an embodiment of the present invention.

FIG. 7 is an eye chart for cataract detection according to an embodiment of the present invention.

FIG. 8A is an eye chart for cataract detection according to an embodiment of the present invention that utilizes a screen to cover a portion of the chart.

FIG. 8B is an eye chart for cataract detection according to the embodiment of the present invention shown in FIG. 8A with the screen in a second position.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

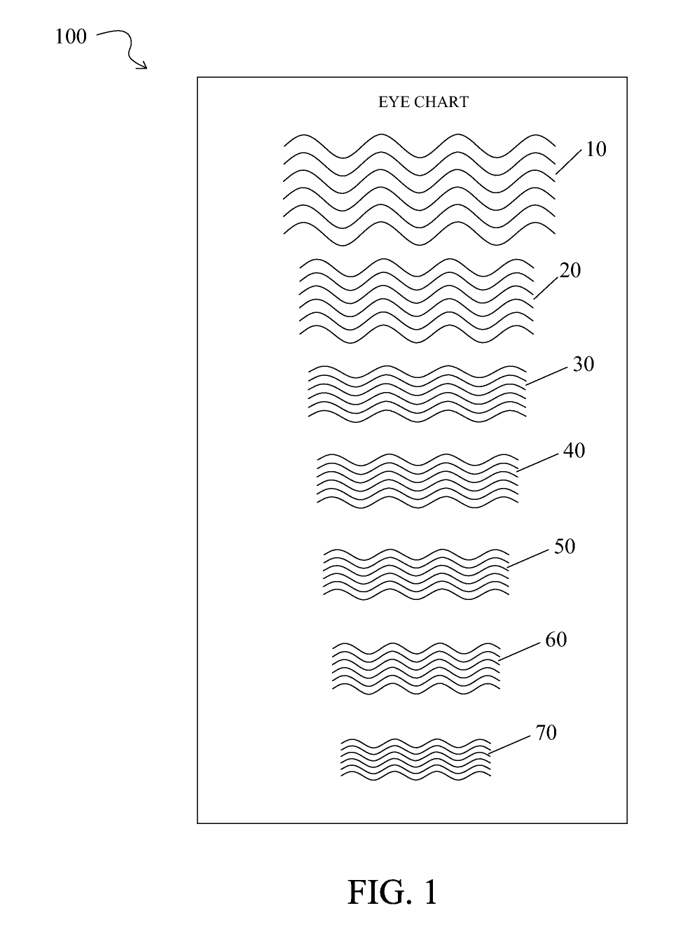

Referring to FIG. 1, a cataract detection eye chart 100 comprises a series of sinusoidal lines. As the user proceeds down the chart 100, the size of the pattern is reduced. Group one lines 10 are spaced 0.3 mm apart and difficulty discriminating at this spacing indicates advanced cataracts. As one proceeds to the group two lines 20, the lines are closer together and are spaced at an interval a user having difficulty discriminating between this spacing indicates progressing cataracts. Likewise group lines three through six; 30, 40, 50 and 60 respectively, reveal progressively lessening cataracts with group seven lines 70 indicating no perceptible cataracts. Of course other spacings may be used and additionally, the spacing may be adjusted for the distance the user will be from the chart 100.

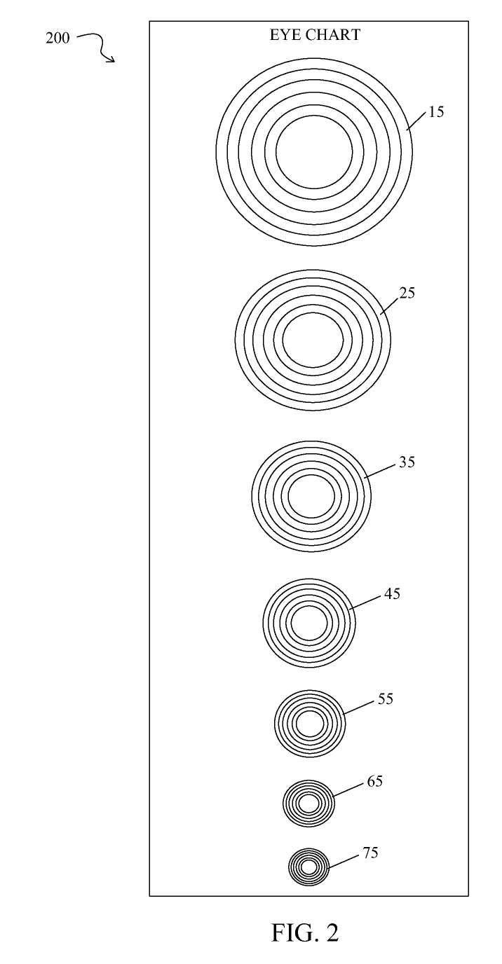

Referring now to FIG. 2, a cataract eye chart 200 is shown having a plurality of circular patterns to indicate the presence of various stages of cataract development. A person only able to discriminate Group one pattern 15 is considered to exhibit an advanced stage of cataract development. Again as in FIG. 1, groups two through six; 25, 35, 45, 55 and 65 respectively, reveal decreasing cataract development with group seven 75 indicating no perceptible cataract development. Again as above, other spacings may be used as well as varying the number of lines used to reduce “expectation error” caused by a user anticipating the number of lines rather than actually reading them.

Of course the number of lines is not critical as long as there are enough lines to present a graphical challenge to discriminate between normal vision and cataract impaired vision. Additionally, the spacing of the lines may be varied.

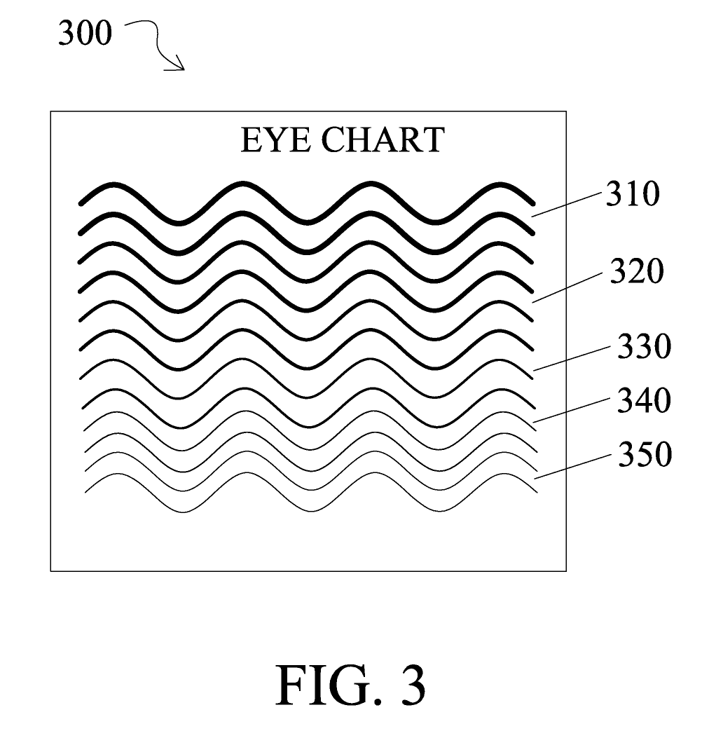

Referring now to FIG. 3, a cataract eye chart 300 is shown having lines grouped together with varying thicknesses. Group one lines 310 have high contrast due to their width and a user only able to discriminate between these lines would be diagnosed as having advanced cataracts. Groups two through four, 320, 330 and 340 respectively, indicate lessening degrees of cataract development with group five 350 indicating unimpaired vision. Again, other contrasting schemes may be utilized such as having as random selection of varying contrasting lines. Additionally, colors could also be used such as having lines in blue or other color to provide an additional level of discrimination. Also, different shades can be used to further refine the optical characteristics such as grey scales where the lines appear from dark to light.

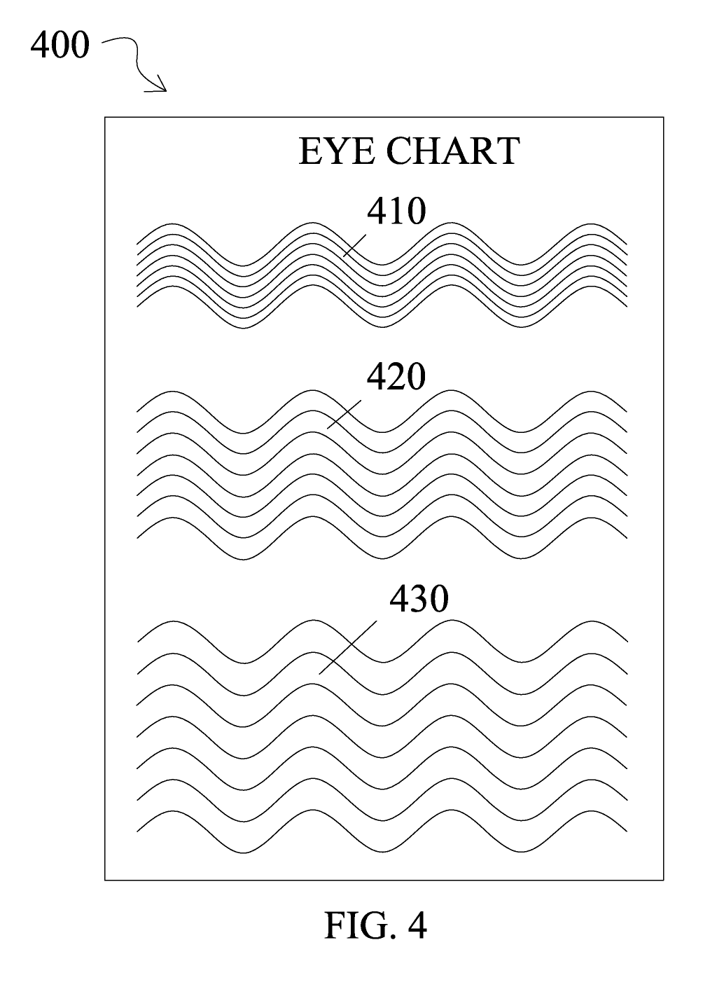

Now referring to FIG. 4, a cataract eye chart 400 is shown with three groups of sinusoidal lines. The first group 410 has 7 lines with small spacings. The second group 420 has 5 lines with intermediate spacings and the third group 430 has six lines with the widest spacings. The number of lines is changed to help avoid an expectation error by a user if the number of lines is the same for all. Of course the number of lines is not critical as long as there are enough lines to present a graphical challenge to discriminate between normal vision and cataract impaired vision.

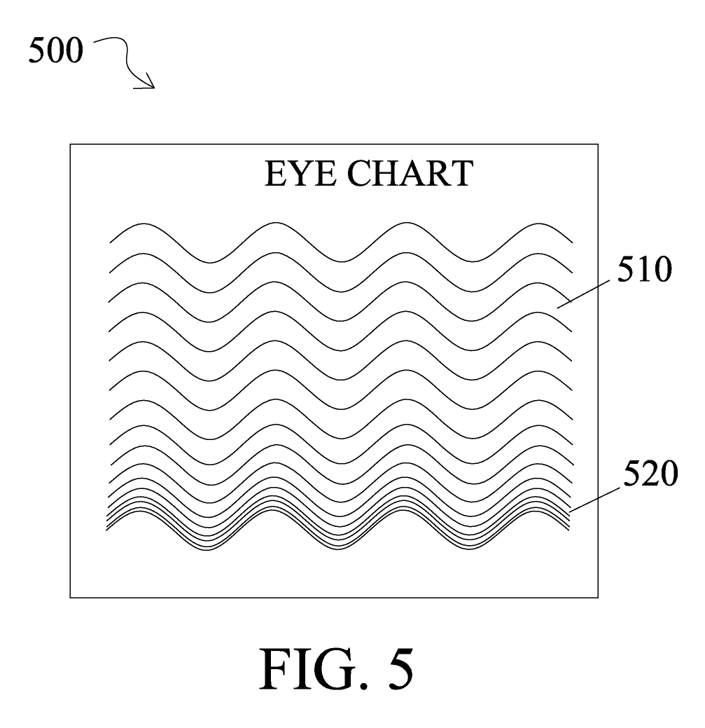

Referring now to FIG. 5, a cataract eye chart 500 is shown having a plurality of sinusoidal lines 510 through 520 that become increasingly closer together. A user is asked to indicate where on chart 500 they are able to discriminate indicating various stages of cataract development. The exact spacing and width of lines may be adjusted depending on the distance from chart 500 the user will be placed.

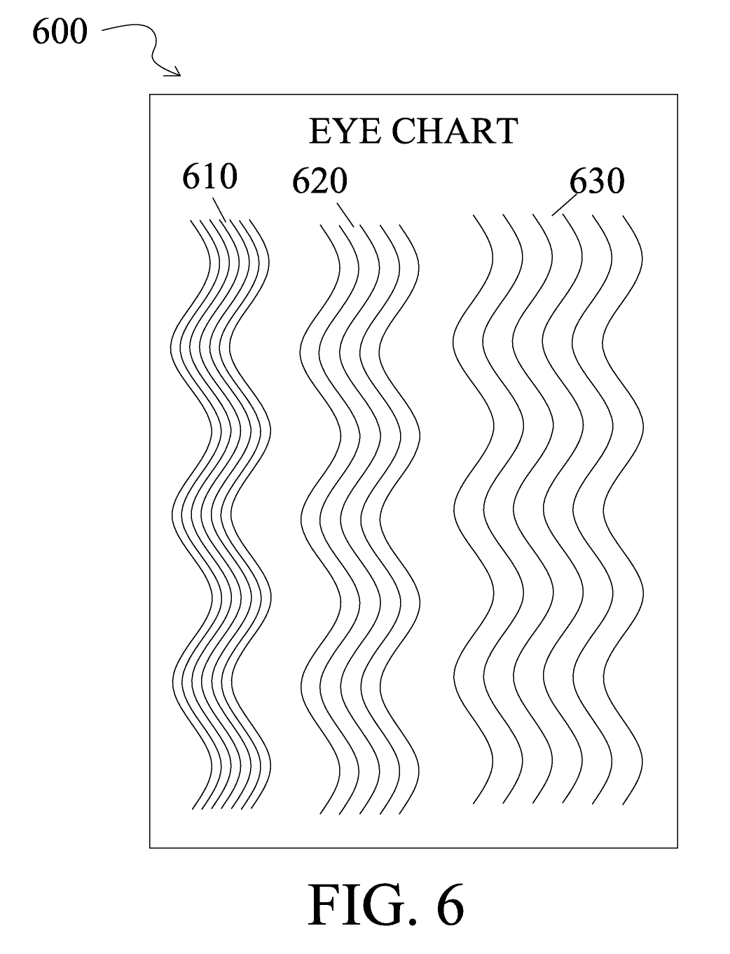

FIG. 6 shows a cataract eye chart 600 having a vertical orientation with three groups of sinusoidal lines. Group one 610 are the closest; second group 620 having an intermediate spacing with the third group 630 being the furthest apart; again as above, discrimination among groups indicate cataract development.

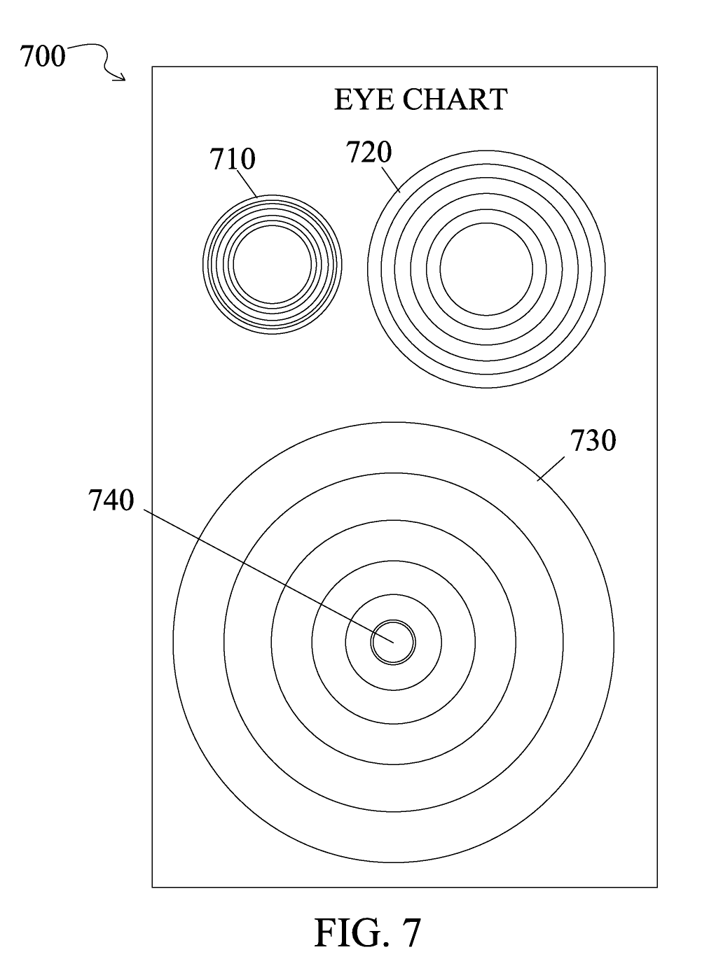

Now referring to FIG. 7, a cataract eye chart 700 is shown having a series of circular patterns to detect and document cataract progression. Group one patterns 710 are the closest together with group two 720 being spaced at an intermediate level and the third group 730 having the widest spacing. Additionally, a light source 740 such as a white light LED or other bright light source may be used to help diagnose cataract development under night driving conditions that may be difficult to detect under normal lighting conditions. Light source 740 may be used with any of the embodiments shown. Of course more or less groups may be used for further levels of discrimination depending on the application.

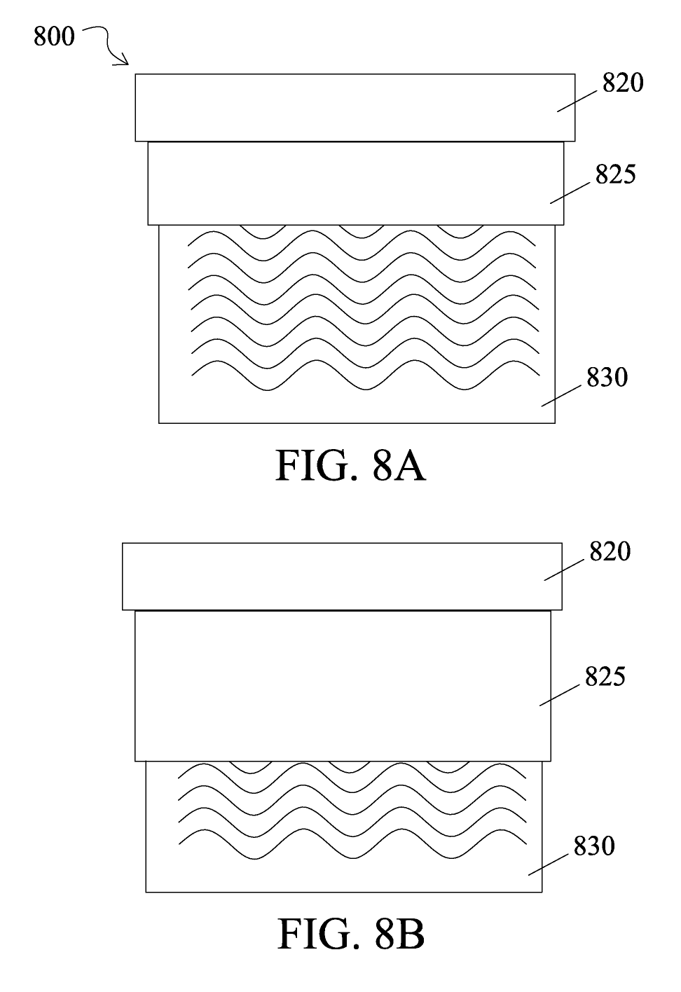

Referring now to FIGS. 8A and 8B, a cataract eye chart 800 is shown with a screen 825 held in a screen holder 820 to cover a portion of an eye chart 830. Screen holder 820 utilizes a retractable mechanism that allows a practitioner to raise and lower the screen as desired while retaining the position as is known in the art. This allows the practitioner to vary the number of lines revealed to reduce expectation error for patients who might remember or pre-count lines to compensate for reduced vision.

If the result of the instant screening reveals a possible problem, the patient may be referred to a specialist for further testing as dictated by standards of care in the health profession. If no problem is indicated, the patient is saved from having to get further testing along with the associated costs. Additionally, the instant method allows a patient to keep track and control the progression of cataracts by recording the results thus allowing the patient and doctor to follow the most advantageous course of treatment.

Although the instant invention has been described in relation to particular embodiments thereof, many other variations and modifications and other uses will become apparent to those skilled in the art.

What is claimed is:

1. An eye chart for screening a patient for possible eye disorders comprising:
a plurality of lines arranged in a pattern adapted to screen said patient for early onset of said possible eye disorder.

2. The eye chart for screening a patient for possible eye disorders according to claim 1 wherein said plurality of lines are arranged in a sinusoidal pattern.

3. The eye chart for screening a patient for possible eye disorders according to claim 1 wherein said plurality of lines are arranged in a circular pattern.

4. The eye chart for screening a patient for possible eye disorders according to claim 1 wherein said plurality of lines are arranged to vary in thickness.

5. The eye chart for screening a patient for possible eye disorders according to claim 1 wherein said plurality of lines are arranged to vary in contrast.

6. The eye chart for screening a patient for possible eye disorders according to claim 1 wherein said plurality of lines are arranged to vary in spacing.

7. The eye chart for screening a patient for possible eye disorders according to claim 1 wherein said plurality of lines are further arranged in a plurality of line groups; and
each of said line groups comprising a grouping of similar lines wherein each said line group varies from another group.

8. The eye chart for screening a patient for possible eye disorders according to claim 7 wherein said grouping of similar lines comprises sinusoidal shaped lines.

9. The eye chart for screening a patient for possible eye disorders according to claim 7 wherein said grouping of similar lines comprises circular shaped lines.

10. The eye chart for screening a patient for possible eye disorders according to claim 7 wherein each said line group varies by being progressively smaller.

11. The eye chart for screening a patient for possible eye disorders according to claim 7 wherein each said line group varies by being comprised of progressively closer lines.

12. The eye chart for screening a patient for possible eye disorders according to claim 7 wherein each said line group varies by being comprised of lines of varying contrast.

13. The eye chart for screening a patient for possible eye disorders according to claim 7 wherein each said line group varies by being comprised of lines of varying thicknesses.

14. The method of screening a patient for possible eye disorders using an eye chart according to claim 1 wherein said possible eye disorder is cataracts.

15. An eye chart for screening a patient for possible eye disorders comprising:
a plurality of lines arranged in a pattern adapted to screen a patient for early onset of cataracts; and
a movable screen for selectively covering at least a portion of said eye chart.

16. The method of screening a patient for possible eye disorders using an eye chart according to claim 15 wherein said possible eye disorder is cataracts.

17. A method of screening a patient for possible eye disorders using an eye chart comprising the steps of:
obtaining an eye chart comprising a plurality of lines arranged in a pattern adapted to screen a patient for early onset of cataracts;
positioning said patient at a selected distance from said eye chart;
requiring said patient to identify a selected characteristic of said eye chart; and
interpreting said patient's identification as an indicator of a possible eye disorder.

18. The method of screening a patient for possible eye disorders using an eye chart according to claim 17 further comprising the step of recording said patient's identification to create a baseline for future evaluations.

19. The method of screening a patient for possible eye disorders using an eye chart according to claim 17 wherein said possible eye disorder is cataracts.

20. The method of screening a patient for possible eye disorders using an eye chart according to claim 1 wherein said plurality of lines are produced in a selected color shade.
